SEQUENCE LISTING (2 sequences)

SEQ ID NO: 1 - DNA, 21 bp, Mus musculus
gaccgctatcaggacatagcg

SEQ ID NO: 2 - DNA, 20 bp, Mus musculus
aggactcaggttctgcctca

(19) United States
(12) Patent Application Publication
(10) Pub. No.: US 2010/0146645 A1
(43) Pub. Date: Jun. 10, 2010

(54) TRANSGENIC ANIMAL MODEL FOR MODELLING PATHOLOGICAL ANXIETY, A METHOD FOR IDENTIFYING COMPOUNDS FOR TREATMENT OF DISEASES OR DISORDERS CAUSED BY PATHOLOGICAL ANXIETY AND A METHOD FOR USING WFS1 PROTEIN AS A TARGET FOR IDENTIFYING EFFECTIVE COMPOUNDS AGAINST PATHOLOGICAL ANXIETY

(76) Inventors: Eero Vasar, Tartu (EE); Sulev Köks, Tartu (EE); Hendrik Luuk, Tartu (EE); Sirli Raud, Tartu (EE); Mario Plaas, Tartu (EE)

Correspondence Address: REED SMITH LLP, P.O. BOX 488, PITTSBURGH, PA, 15230-0488, US

(21) Appl. No.: 12/518,422

(22) PCT Filed: Dec. 10, 2007
(86) PCT No.: PCT/EE2007/000025

§ 371 Date: Nov. 12, 2009

(30) Foreign Application Priority Data
Dec. 12, 2006 (EE) P200600039

(51) Int. Cl. G01N 33/68; A01K 67/027; G01N 33/00

(52) U.S. Cl. 800/3; 800/9; 436/501

(57) ABSTRACT

The invention discloses the transgenic animal model for pathological anxiety, the method to generate this model, the method to test drugs and drug candidates for the treatment of pathological anxiety and the method to use Wfs1 as target for screening of new anxiolytic drugs to treat pathological anxiety. This animal model is useful to test potential drug candidates for the treatment of diseases caused by pathological anxiety and to screen therapeutic compounds for the psychiatric disorders caused by reduces stress-tolerance and deficiency in adaptation to environmental challenges.

### CROSS-REFERENCE TO RELATED APPLICATIONS

This application is a national phase application pursuant to 35 U.S.C. §371 of International Application No. PCT/EE2007/000025, filed Dec. 10, 2007, which claims priority to Estonia Application No. P200600039, filed Dec. 12, 2006.

### FIELD OF THE INVENTION

The present invention relates to the field of molecular biology.

More particularly, the invention relates to transgenic animals that can serve as models for psychological disorders caused by pathological anxiety. Pathological anxiety causes the reduction of the ability of the organism to adaptations in stressful conditions and reduction of general coping.

### BACKGROUND OF THE INVENTION

The current invention relates to Wfs1 gene and anxiety disorders, as an example we describe Wfs1 deficient mice as a model for anxiety disorders.

Anxiety disorders are among the most prevalent psychological disorders and their treatment requires significant expenses from the health care system. For example, in the USA it has been found that about 25% of the population suffers from some kind of anxiety disorder during some life stage and the treatment of these patients costs about 44 million dollars per year (Greenberg et al., 1999; Hettema et al., 2001; Kessler et al., 1994). Anxiety is an emotion, which is connected to the response of the organism to stressogenic factors, whereas the hazardous factors are potential and avoidable. Anxiety becomes pathological, when the accompanying reactions are excessive or the duration of the state of anxiety is too lengthy. Anxiety disorders are classified as stress-related or stress non-related anxiety disorders. Stress-related anxiety disorders involve adaptation disorder, acute stress response; stress non-related anxiety disorders are panic disorder and generalized anxiety disorder. Anxiety can be studied using animal models. Animal models exist, where trait anxiety or state anxiety has been induced in lab animals. For example, U.S. Pat. No. 6,353,152 (Vale Wylie. W., Lee Kuo-Fen, Bale Tracy L., Smith George W., Corticotropin releasing factor receptor 2 deficient mice and uses thereof) and U.S. Pat. No. 6,060,642 (Tecott Laurence H., Brennan Thomas J., Serotonin 5-HT6 receptor knockout mouse) represent animal models for anxiety.

Wolfram syndrome is a rare hereditary genetic disorder caused by loss-of-function mutations in the Wfs1 gene. This disorder is sometimes referred to as DIDMOAD (diabetes insipidus, diabetes mellitus, optic atrophy and deafness). Juvenile diabetes mellitus and optic atrophy are most commonly described early symptoms of this hereditary disorder (Wolfram D J, Wagener H P (1938) Diabetes mellitus and simple optic atrophy among siblings: report of four cases. Mayo Clin Proc 13:715-718). In addition to abnormalities in endocrine system, Wolfram syndrome patients develop various neurodegenerative symptoms with optic atrophy, hearing loss, nystagm, peripheral neuropathy and dementia among them (Swift R G, Sadler D B, Swift M (1990) Psychiatric findings in Wolfram syndrome homozygotes. Lancet 336:667-669; Swift R G, Perkins D O, Chase C L, Sadler D B, Swift M (1991) Psychiatric disorders in 36 families with Wolfram syndrome. Am J Psychiatry 148;775-779). Wolfram syndrome has multisystem manifestations whereas diabetes mellitus and diabetes insipidus strongly suggest involvement of the endocrine system. The necessary symptoms for the diagnosis of Wolfram syndrome are juvenile insulin dependent diabetes and bilateral progressive optic atrophy. Both may be present in childhood, adolescence, or early adult life; typically, but not invariably, diabetes mellitus is detected first. The diabetes occurring in case of Wolfram syndrome can be distinguished from the juvenile diabetes by the absence of the antibodies against glutamate decarboxylase (GAD-65). These antibodies have been described in Wolfram syndrome only in single cases. Among the neurological symptoms in Wolfram syndrome patients hearing loss, urinary tract atony, ataxia, peripheral neuropathy, mental retardation, dementia, and psychiatric illnesses should be outlined. Moreover, widespread atrophic changes in the brain of Wolfram syndrome patients have been described (Rando T A, Horton J C, Layzer R B (1992) Wolfram syndrome: evidence of a diffuse neurodegenerative disease by magnetic resonance imaging. Neurology 42:1220-1224). It has been shown, that 60% of Wolfram syndrome patients have episodes of severe depression, psychosis, or organic brain syndrome, as well as compulsive verbal and physical aggression (Swift R G, Sadler D B, Swift M (1990) Psychiatric findings in Wolfram syndrome homozygotes. Lancet 336;667-669). Estimated risk for a Wolfram syndrome heterozygote to be hospitalized for psychiatric illness or to attempt suicide is approximately 8 times higher than that of a non-carrier (Swift R G, Perkins D O, Chase C L, Sadler D B, Swift M (1991) Psychiatric disorders in 36 families with Wolfram syndrome. Am J Psychiatry 148:775-779).

Importance of wolframin gene in predicting risk for mood disorders was verified recently (Koido K, Koks S, Nikopensius T, Maron E, Altmae S, Heinaste E, Vabrit K, Tammekivi V, Hallast P, Kurg A, Shlik J, Vasar V, Metspalu A, Vasar E (2005) Polymorphisms in wolframin (WFS1) gene are possibly related to increased risk for mood disorders. Int J Neuropsychopharmacol 8:235-244). However, the role and detailed mechanism of Wfs1 protein in the development of mood disorders and other psychiatric disorders is unknown yet.

In U.S. Pat. No. 6,984,771 (Mice heterozygous for WFS1 gene as mouse models for depression, Roberds, Steven L, Huff, Rita M., 2006; “the '771 patent) a recombinant depression model in rodents has been described. The rodent disclosed in the '771 patent has cells with mutations appearing in the Wfs1 gene. The described rodent is a mouse heterozygous for mutations in the 8th exon of the Wfs1 gene. Due to the mutations, a non-functional wolframin protein is obtained, which lacks all or part of the transmembranic regions. In the named patent methods and descriptions for making and using such mouse and its cells are disclosed.

Methods for the assessment of the activity of the wolframin protein have been disclosed in US patent application US20040058405 (Pharmacia & Upjohn Company, 2004) and U.S. Pat. No. 7,037,695 (Pharmacia & Upjohn Company, 2006).

The aforementioned patents and applications are related to the wolframin protein and the method of assessment of the modulators of the interaction of it and its binding to appropriate cellular partner.

WFS1 isolated from human chromosome 4p has been disclosed in international patent application PCT/US99/22429 (WO0018787, Washington University, Permutt, M. Alan, et al, 2000). The association of WFS1 gene mutation with the development of Wolfram syndrome has been described. The authors have suggested that the WFS1 gene together with its cDNAs, encoded protein and antibodies immunologically specific for it, may represent a biological marker for early diagnosis of the syndrome and assessment of a persons predisposition for this syndrome.

### SUMMARY OF THE INVENTION

The current invention comprises an animal model for pathological anxiety and methods for using it, including 1) methods for using the described animal model for the assessment of the efficiency of substances and therapeutical agents useful for treatment of disorders caused by pathological anxiety; 2) methods for using the described animal model for the assessment of substances and therapeutical agents, which increase the expression of the Wfs1 protein and suppress pathological anxiety; 3) methods for using the Wfs1 protein as a target for testing the efficiency of drugs or other therapeutical compounds for the treatment of disorders caused by pathological anxiety.

The present invention provides an animal model for pathological anxiety, which comprises a rodent without a functional Wfs1 protein or with a Wfs1 protein of impaired properties, wherein the rodent lacks both of the wild type alleles of the Wfs1 gene or wherein the function of the Wfs1 protein of the rodent is impaired for example via suppressing the expression level by RNA interference with antisense oligo- or polynucleotides or—nucleotide analogues or wherein the rodent does express a compound protein, which has been created by substituting a part of the genomic coding sequence of the wild type Wfs1 gene with a coding sequence of an identifyable marker gene. An ordinarily skilled artisan will recognize that the identifyable marker protein may be any protein (enzyme, fluorescent protein, affinity target), which has been appended in reading frame to the sequence encoding the Wfs1 protein or which is controlled by the DNA regulatory elements regulating the expression of the Wfs1 gene and which can be conveniently visualized in the tissues and/or cells of the animal of interest. Preferably, the identifiable marker protein of the invention is the β-galactosidase enzyme (LacZ protein), which can be visualized by using standard LacZ staining procedures.

The distinctive properties and expressions of the proposed animal model for pathological anxiety include: 1) increased sensitivity to stress—some mice express vocalization in connection with environmental changes or in connection with getting into a new environment, as well as vocalization to mice vocalizing in another cage; 2) reduced exploratory activity; 3) increased frequency of risk avoidance behaviours.

In case of the transgenic model for pathological anxiety we are dealing with a rodent with noticeable difficulties in adaptation to the environment, increased stress sensitivity and several symptoms of anxiety. One embodiment of an animal model is a mouse lacking both of the wild type Wfs1 alleles and exhibiting complete lack of the function of the Wfs1 protein. These mice exhibit a behaviour similar to anxiety disorder in models based on inherited anxiety responses (significantly reduced explorative behaviour in elevated plus-maze, light-dark cage exploration test, motor activity box, increased risk behaviour in elevated plus-maze, and avoidance of novel food in hyponeophagia test). Suppressed exploratory activity and risk behaviour were significantly reduced with diazepam (1 mg/kg), a GABAA receptor agonist, which is a clinically widely used medication against anxiety. Increased sensitivity of Wfs1-deficient mice to the anxiolytic action of diazepam can be related to changes in activity of GABAergic system.

Experimentally naive Wfs1-deficient animals display a significant down-regulation of α1 (Gabra1) and α2 (Gabra2) subunits of GABA_A receptors, mediating sedative and anxiolytic effect of diazepam, in the temporal lobe and frontal cortex. Similar changes occur in the same brain areas of wild-type mice when wild-type mice are exposed to the elevated plus-maze. Since the expression of enzymes responsible for the synthesis of GABA is not significantly affected by the invalidation of Wfs1 gene, then the increased anxiety established in Wfs1-deficient mice as well as the increased anxiolytic action of diazepam could be linked to down-regulation of GABA_A receptor subunits. These mice exhibit difficulties in adaptation to new environment or changes in environment (new cage, new room, transportation). Moreover, in stressogenic situations some of these mice exhibit peculiar vocalization (audible sound or whistle). Such vocalization depends on the level of stress. In a room with dim light (20 lux) the transgenic mice exhibit a vocalization resembling bird warbling in elevated plus-maze test. In a room with very bright lighting (1000 lux) the vocalization of these mice intensifies and resembles a creak from a door. Wild type and heterozygous mice do not make any sound in a similar situation. Some animals vocalize also in reply to the companions in the neighbouring cage, who create similar sounds. Thus it is possible to assess the tolerance of the Wfs1 mutant homozygous mice to significantly lower environmental changes. Stress-induced vocalization in Wfs1 −/− mice could be removed by administering 1 mg/kg diazepam. A lower dose 0.5 mg/kg of diazepam is not effective. Accordingly, the present animal model can be used for testing new drugs for the treatment of pathological anxiety. The current invention is the first animal model, which is a mouse model for pathological anxiety created using gene technology, that expresses difficulties in adaptation with environment, increased stress sensitivity and other symptoms of anxiety.

The current invention involves methods for the identification of compounds useful for the treatment of psychological disorders, which are at least partially caused by pathological anxiety and which comprise administering one or more agents under testing to a rodent, who lacks functional Wfs1 protein or whose function of the Wfs1 protein is disturbed. The efficiency of potential anxiolytics in removing/reducing the symptoms of anxiety is established by comparing the effect of the investigated compound with the effect of diazepam, the classical anti-anxiety drug. The symptoms and markers of anxiety comprise: 1) increased stress sensitivity; 2) reduced exploratory activity; 3) increased risk avoidance behaviour.

The invention consideres methods for using the Wfs1 protein as a target for identifying compounds eliminating pathological anxiety, which comprise administration of one or more agent under investigation to rodents, who lack functional Wfs1 protein or to animals with reduced levels of functional Wfs1 protein. Likewise, the screening for new anti-anxiety substances comprises studies, where the expression level of the Wfs1 protein is determined in parallel with reduction or disapperence of behavioural symptoms of anxiety. As one option, primary antibodies against wild type Wfs1 protein can be used to observe Wfs1 expression level. As another option, the activity of an identifiable marker protein appended by homologous recombination to the sequence encoding the Wfs1 protein or a identifiable marker protein under the transcriptional control of the Wfs1 gene promoter is used as the indicator of Wfs1 protein expression level.

### Definitions

Pathological Anxiety

As used herein, the term “pathological anxiety” refers to a chronic condition, where excessive anxiety occurs in case of lack of real threats, causing reduction of the capability of an individual to cope with problems, suppresses motivation and induces the status of constant stress and exhaustion. On the contrary to pathological anxiety, normal anxiety is an adaptation-promoting mechanism, which increases the readiness of an individual to cope with demanding or potentially dangerous situations.

Wfs1 Protein or Wolframin

As used herein, the term “Wfs1 protein” refers to a human protein of 890 amino acids that has an amino acid sequence as described in, for example, (Inoue H, Tanizawa Y, Wasson J, Behn P, Kalidas K, Bernal-Mizrachi E, Mueckler M, Marshall H, Donis-Keller H, Crock P, Rogers D, Mikuni M, Kumashiro H, Higashi K, Sobue G, Oka Y, Permutt M A (1998), which is incorporated herein by reference. A gene encoding a transmembrane protein is mutated in patients with diabetes mellitus and optic atrophy (Wolfram syndrome). Nat Genet 20:143-148) and (Strom T M, Hortnagel K, Hofmann S, Gekeler F, Scharfe C, Rabl W, Gerbitz K D, Meitinger T (1998) Diabetes insipidus, diabetes mellitus, optic atrophy and deafness (DIDMOAD) caused by mutations in a novel gene (wolframin) coding for a predicted transmembrane protein. Hum Mol Genet 7:2021-2028), and other mammalian homologs thereof, such as described in (Takeda K, Inoue H, Tanizawa Y, Matsuzaki Y, Oba J, Watanabe Y, Shinoda K, Oka Y (2001). WFS1 (Wolfram syndrome 1) gene product: predominant subcellular localization to endoplasmic reticulum in cultured cells and neuronal expression in rat brain. Hum Mol Genet 10:477-484) (rat homologue). Exemplary proteins intended to be encompassed by the term “Wfs1 protein” include those having amino acid sequences disclosed in GenBank with accession numbers NP_{—}005996, NP_{—}005996.1, CAA77022, AAH30130.1, AAC64943, AAH30130, CAA77022.1, AAC64943.1 or e.g., encoded by nucleic acid molecules such as those disclosed in GenBank with accession numbers NM_{—}006005.2 (gi:1337699)], Y18064.1 (gi:3766440), BC030130.2 (gi:33871564), AF084481.1 (gi:3777582), NM_{—}031823.1 (gi:13929175), AF136378.1 (gi:7381176), NM_{—}011716.1 (gi:6755996), AJ011971.1 (gi:3776089), BC046988.1 (gi:28422738), AF084482.1 (gi:3777584). Wfs1 is also referred to in the art as Wolfram syndrome 1, Wolframin, WFS, DFNA6, DFNA14, DFNA38, DIDMOAD.

Markers of Anxiety

Various numerical values or scores used for the assessment of the anxiety of the experimental animal or a test subject. To obtain a score or value for assessing anxiety specific behavioural tests are used, but observing spontaneous behaviour may also be sufficient.

### BRIEF DESCRIPTION OF THE DRAWINGS

FIG. 1 Schematical presentation of the construct used for knocking out the gene encoding Wfs1 protein.

FIG. 2 and FIG. 3. Exploratory activity of female and male Wfs1 −/− mice in elevated plus-maze.

FIG. 2. Time spent on an open arm (s). White bars—female wild type mice; black bars—female Wfs1 −/− mice; vertical striped bars—male wild type mice; vertical striped bars—male Wfs1 −/− mice. *p<0.05 compared to female Wfs1 −/− mice. +p<0.05 compared to male Wfs1 −/− mice (Newman-Keuls test, Two-way ANOVA).

FIG. 3 Number of head dippings from the open arm. White bars—female wild type mice; black bars—female Wfs1 −/− mice; vertical striped bars—male wild type mice; vertical striped bars—male Wfs1 −/− mice. &p<0.05 compared to female wild type Wfs1 −/− mice. **p<0.05 compared to female wild type Wfs1 −/− mice (Newman-Keuls test, Two-way ANOVA).

FIG. 4, FIG. 5 and FIG. 6 The effect of diazepam (1 mg/kg) on the exploratory activity of female Wfs1 −/− mice in elevated plus-maze.

FIG. 4 Time spent on the open arm (s). White bars—wild type mice administered with physiological saline; checked bars—wild type mice administered with (1 mg/kg) diazepam; black bars—Wfs1 −/− mice administered with physiological saline; diagonally striped bars—Wfs1 −/− mice administered with (1 mg/kg) diazepam.

FIG. 5 Number of head dippings from the open arm. White bars—wild type mice administered with physiological saline; checked bars—wild type mice administered with (1 mg/kg) diazepam; black bars—Wfs1 −/− mice administered with physiological saline; diagonally striped bars—Wfs1 −/− mice administered with (1 mg/kg) diazepam. *p<0.05 compared to wild type mice administered with physiological saline. (Newman-Keuls test, Two-way ANOVA).

FIG. 6 Risk avoidance behaviour. White bars—wild type mice administered with physiological saline; checked bars—wild type mice administered with (1 mg/kg) diazepam; black bars—Wfs1 −/− mice administered with physiological saline; diagonally striped bars—Wfs1 −/− mice administered with (1 mg/kg) diazepam. *p<0.05 compared to wild type mice administered with physiological saline. +p<0.009 compared to Wfs1 −/− mice administered with physiological saline. (Newman-Keuls test, Two-way ANOVA).

FIG. 7, FIG. 8 and FIG. 9. Exploratory activity of female and male Wfs1 −/− mice in light-dark cage.

FIG. 7 Entries into the third region. White bars—female wild type mice; black bars—female Wfs1 −/− mice; vertical striped bars—male wild type mice; grey bars—male Wfs1 −/− mice. *p<0.05 compared to female wild type mice. (Newman-Keuls test, Two-way ANOVA).

FIG. 8 Time spent in the light region (s). White bars—female wild type mice; black bars—female Wfs1 −/− mice; vertical striped bars—male wild type mice; grey bars—male Wfs1 −/− mice. *p<0.05 compared to female wild type mice; +p<0.05 compared to female Wfs1 −/− mice; +++p<0.05 compared to male Wfs1 −/− mice. (Newman-Keuls test, Two-way ANOVA).

FIG. 9 Number of rises on back paws. White bars—female wild type mice; black bars—female Wfs1 −/− mice; vertical striped bars—male wild type mice; grey bars—male Wfs1 −/− mice. *p<0.05 compared to female wild type mice; **p<0.01 compared to male wild type mice. (Newman-Keuls test, Two-way ANOVA).

FIG. 10, FIG. 11, FIG. 12 and FIG. 13 exploratory activity of female Wfs1 −/− mice in motor activity box.

FIG. 10 Time spent in the middle (s). White bars—wild type mice; black bars—Wfs1 −/− mice; *p<0.05 compared to wild type mice. (Tukey HSD test, One-way ANOVA).

FIG. 11 Number of rises on back paws. White bars—wild type mice; black bars—Wfs1 −/− mice. *p<0.05 compared to wild type mice. (Tukey HSD test, One-way ANOVA).

FIG. 12 Motility in cage (m). White bars—wild type mice; black bars—Wfs1 −/− mice. **p<0.01 compared to wild type mice. (Tukey HSD test, One-way ANOVA).

FIG. 13 Duration of motility in cage (s). White bars—wild type mice; black bars—Wfs1 −/− mice. **p<0.01 compared to wild type mice. (Tukey HSD test, One-way ANOVA).

FIG. 14 Hyponeophagia test. White bars—female wild type mice; black bars—female Wfs1 −/− mice. *p<0.05 compared to wild type mice. (Tukey HSD test, One-Way ANOVA).

FIG. 15 and FIG. 16 LacZ staining to present the expression level of Wfs1 gene and its determination.

### DETAILED DESCRIPTION OF THE PREFERRED EMBODIMENTS

### Example 1

### Creating a Transgenic Animal Model for Pathological Anxiety

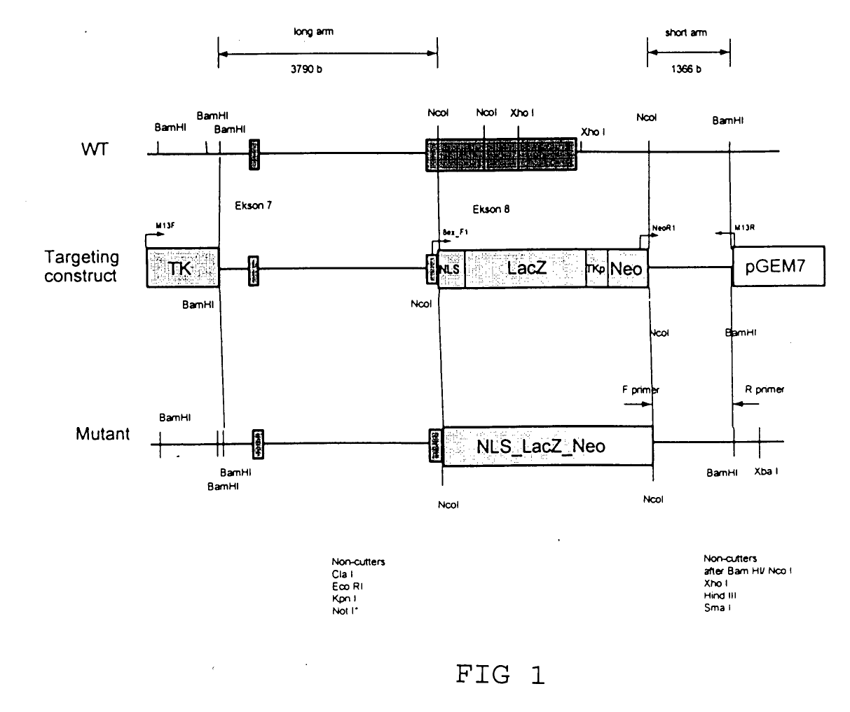

The ordinarily skilled artisan recognizes that there are a number of basic strategies for creating a rodent with nonfunctional Wfs1 protein. In a preferred embodiment a mutation is introduced into the wild-type Wfs1 gene of a rodent so that it renders the Wfs1 protein nonfunctional. In a preferred embodiment this is done by cloning a DNA targeting construct that comprises a mutation (point mutation, deletion, insertion) flanked (e.g. surrounded) by sequences of desired length of the wildtype Wfs1 gene allele to permit homologous recombination (FIG. 1). In preferred embodiments, the rodent is the mouse as mouse is the only mammal where homologous recombination with efficient germline transmission is currently available. An exemplary procedure used in the present invention to generate a transgenic mouse line expressing nonfunctional Wfs1 protein and having a NLS-LacZ marker protein fused to the truncated form of Wfs1 polypeptide includes the following steps: 1) A 500 bp PCR product from the 8th exon of mouse WFS1 gene was used as a probe to screen mouse genomic PAC library RPCI21 (derived from 129/SvevT ACfBR mouse DNA), as a result clone 391-J24 was isolated. 2) A 9.7 kb BamHI fragment was isolated from clone 391-J24 including the 7th and 8th exons of the Wfs1 gene with flanking introns and the named fragment was subcloned into pGem-11Z+ (Promega) cloning plasmid. 3) A 3.7 kb NcoI fragment containing all but the first 208 nucleotides of the eighth exon of the Wfs1 gene and 1.3 kb of the following noncoding genomic sequence was replaced with an in-frame NLSLacZNeo cassette. 4) pgk-TK negative selection cassette was cloned via XhoI into pGem11-Z+ multicloning site upstream of the 5′ genomic arm of the targeting construct. 5) The Wfs1 targeting construct was transformed into DH-5α E. coli competent cells and purified from bacterial lysates using Plasmid Midi Kit (QIAGEN). 6) 40 μg of Wfs1 targeting construct was linearized with NotI and precipitated in cold ethanol. 7) 20 μg of the targeting construct was electroporated into W4/129S6 embryonic stem cells (Taconic) and positive clones were selected using G418 selection. Positive clones were enriched for homologous recombination events using resistance to Gancyclovir treatment. 8) All surviving embryonic stem cell clones were picked and genotyped for homologous recombination by PCR using primers NeoRI (5′-gac cgc tat cag gac ata gcg-3′; SEQ ID No. 1) and Wfs1_WTR1 (5′-agg act cag gtt ctg cct ca-3′; SEQ ID No. 2) and verified with DNA sequencing. 9) Clones identified as positive for homologous recombination were injected into C57/bl6 blastocysts to obtain chimeric mice. 10) Male chimeras were mated with wildtype C57/bl6 mice or 129/SvEvTac mice to obtain mice heterozygous for Wfs1 deficiency. 11) Mice homozygous for Wfs1 deficiency were obtained by mating heterozygotes.

The expression of the Wfs1 protein could be reduced in transgenic animals also by means of RNA interference with antisense oligo- or polynucleotides or nucleotide analogs. In that case antisense construct is inserted into the genome of the transgenic mouse and this insertion is inheritable.

### Example 2

### Animal Models for Pathological Anxiety

In order to describe some possible applications of the object of the invention, we performed several behavioural experiments. Anxiety markers were assessed or animal behaviour was scored in the experiments.

Anxiety Tests.

Ethological models based on inborn anxiety reactions.

Elevated Plus-Maze

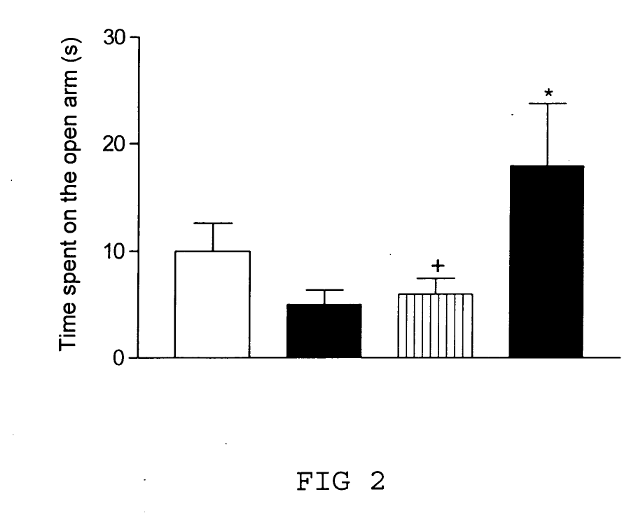

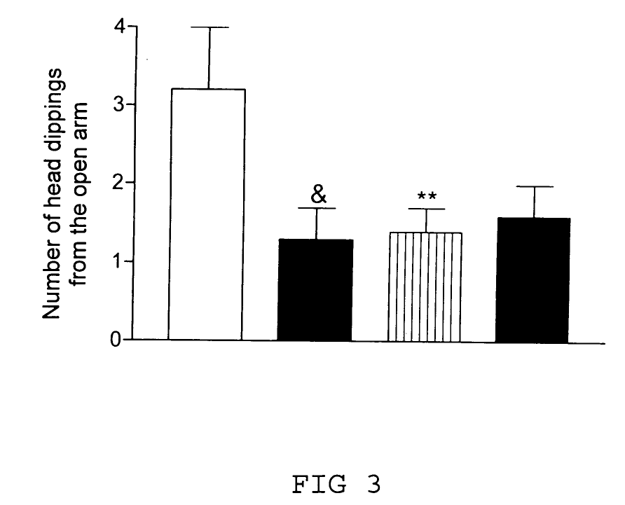

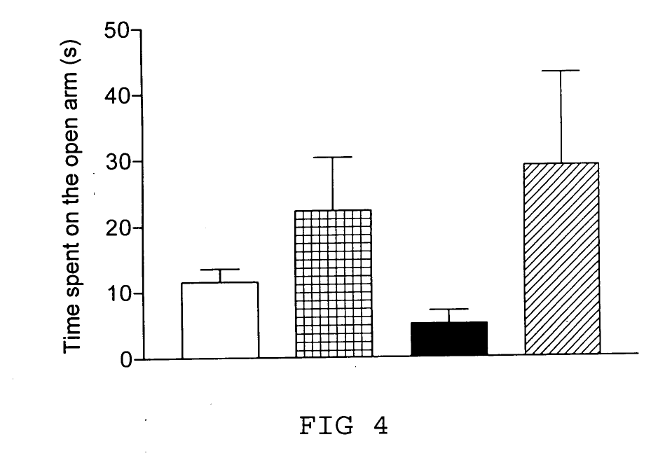

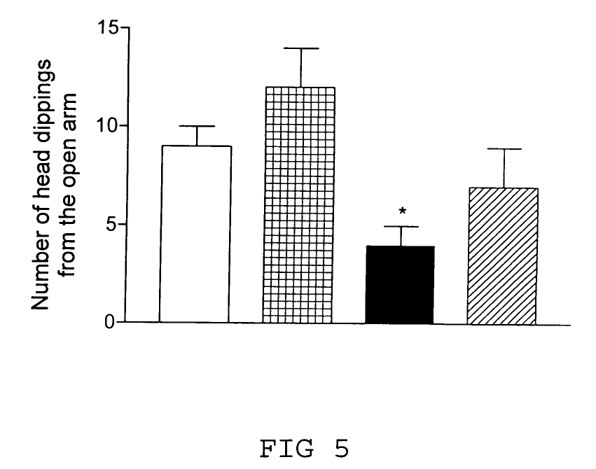

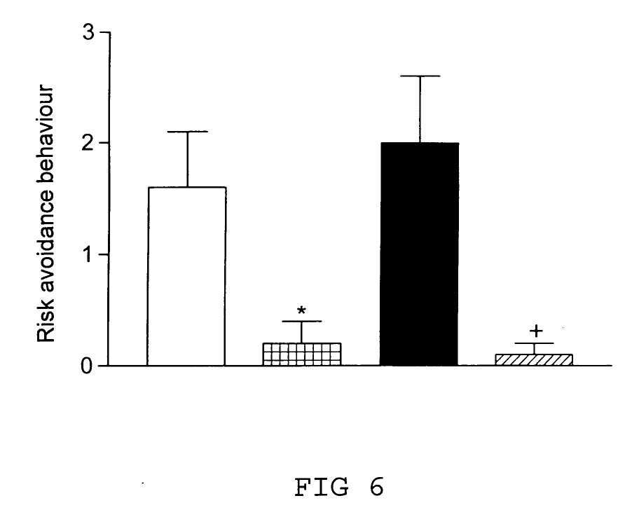

The plus cage consisted of two reciprocally positioned closed (surrounded by walls) and open arms, resembling a plus sign in shape. The cage was elevated to the height of 30 cm. The principle of the model consists in the tendency of anxious animals to avoid entering the open arms of the cage and to prefer to stay in the closed arms. The experiment was performed with preceding isolation (15-20 min) of the animals from their cage fellows. Lighting level during the experiment was 12-20 lux. The experiments revealed that female and male Wfs1 −/− mice behave differently. Namely, female Wfs1 −/− mice exhibit a significant reduction of the exploratory activity (anxiety) and the males display an increase in exploratory activity. Compared to female wild type mice, the female Wfs1 −/− animals spent 2 times less time on the aversive open arm (FIG. 2) and performed downwards examinations from the open arm 1.6 times less (FIG. 3). We have observed such kind of behavioural distinction between male and female mice among wild type mice after three weeks of isolation (Abramov et al., 2004). Conclusively, the Wfs1 −/− mice are extremely sensitive to environmental factors. Short isolation causes similar behavioural changes in transgenic animals like long-term isolation in their wild-type littermates. Diazepam increased the time spent on the open arms of the cage in female Wfs1 −/− homozygotes (FIG. 4), the number of downwards examinations from the open arms (FIG. 5), while risk avoidance behaviour significantly decreased (FIG. 6) and vocalization terminated (vocalization: physiological saline group 24%, diazepam group 0%). An interesting influence on the vocalization of the mice occurred also due to applying lighting levels of different intensities. Namely, at low lighting intensity 19% of the Wfs−/− animals vocalized, while at very strong lighting the amount of vocalizing mice with mutated Wfs1 gene grew to 24% together with significant increase in the intensity of the vocalization. We discovered weight differences between different genotypes. The average weight of the male mice of the “wild type” was 28.6 g, that of Wfs1 −/− of the same sex was 22.5 g; “wild type” females 23.8 g and Wfs1 −/− 19.9 g.

Light-Dark Cage Test

The cage was divided into two: a ⅔ part was lighted and a ⅓ part with darkened cover. The light part was divided into three equal parts, so that the most aversive part was the third part, which is located most distantly from the dark part. Anxious animals preferred to remain mostly in the dark part and avoided the aversive light partition. The experiment was performed one week after the elevated plus-maze experiment and no preliminary isolation of the animals was applied. Lighting level in the light part of the cage was 270 lux.

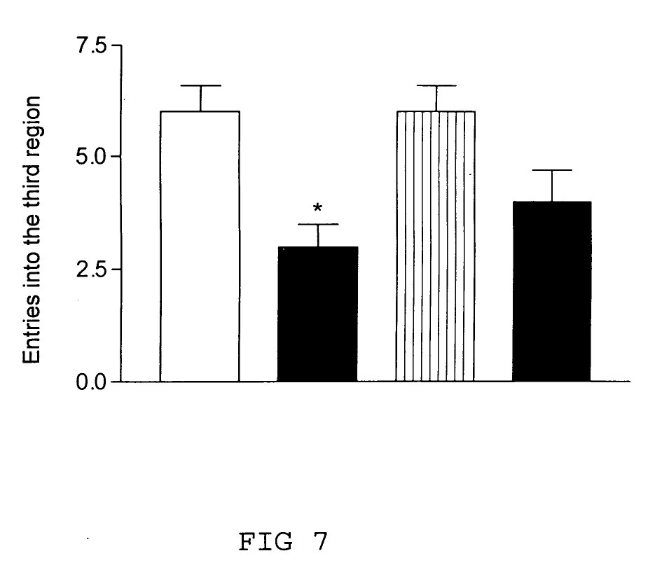

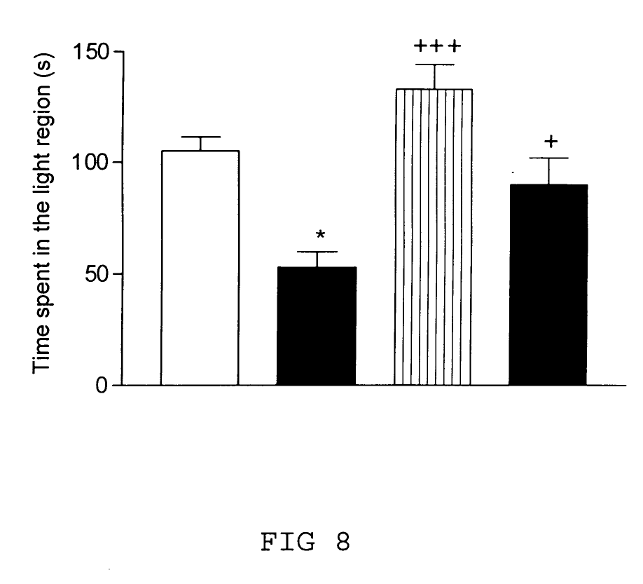

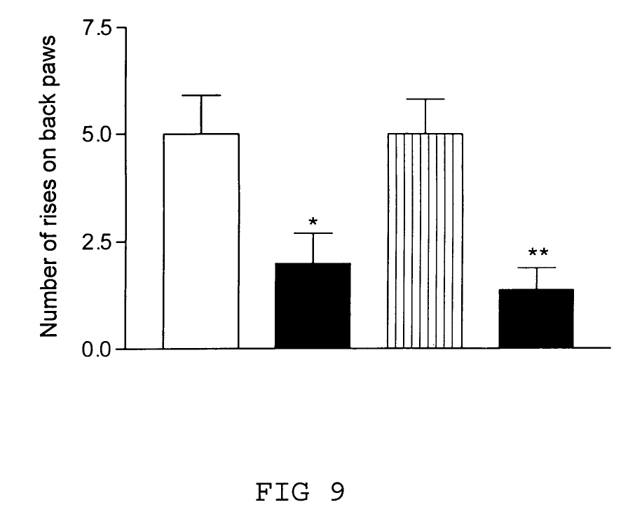

The results of the experiment revealed that both female and male Wfs1 −/− mice exhibited anxiety-like behaviour, while it was somewhat more clearly expressed in females. The Wfs1 −/− mice were significantly more anxious than the wild type mice. Namely, the female Wfs1 −/− mice performed 1.5-2 times less entrances to the various parts of the light partition, if compared to the wild type mice (FIG. 7) and the duration of their stay in the light part was twice shorter (FIG. 8). In addition, the Wfs1 −/− mice exhibited 2.5 times less rises on hindpaws, which also reflects the anxiety of the animal (FIG. 9). In the test cage 30% of the Wfs1 −/− mice vocalized. Injecting an anxiolytic dose of diazepam completely removed the vocalization.

Motor Activity Box

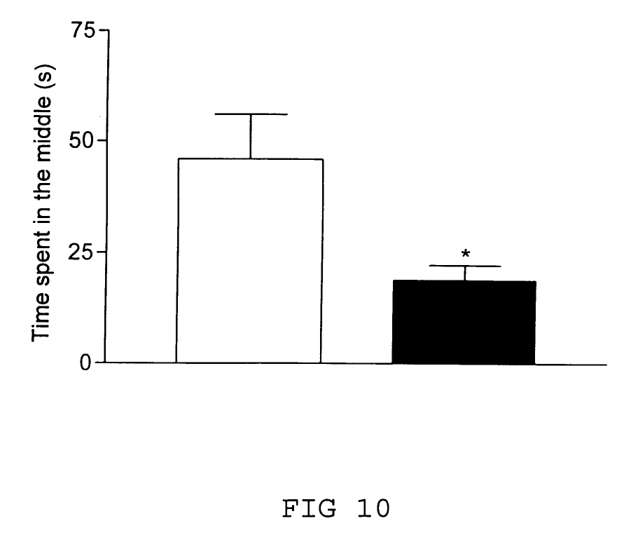

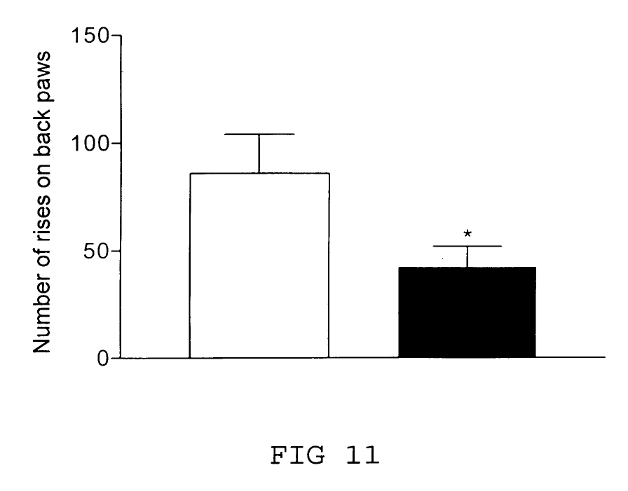

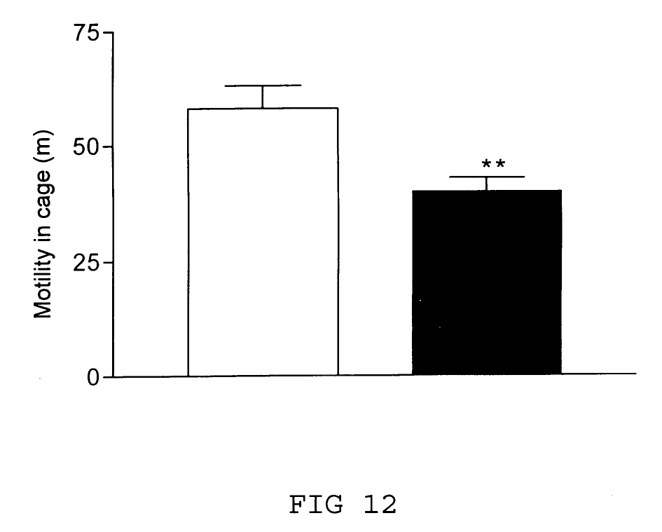

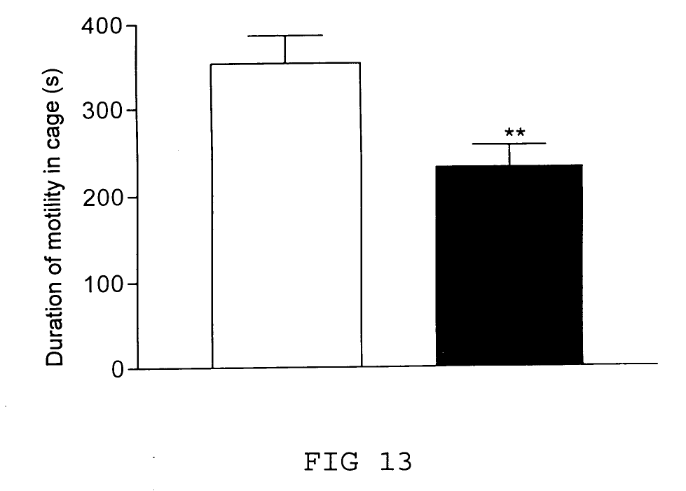

Mice were studied for exploratory activity during 30 minutes in a cage supplied with photosensors (448×448×450 mm). No difference was found between the male mice, female Wfs1 −/− mice exhibited significantly reduced exploratory activity. Wfs1 −/− mice spent 2.5 times shorter periods in the middle of the cage compared to the wild type mice (FIG. 10). Wfs1 −/− mice made 2 times less rises on hindpaws (FIG. 11). Different genotypes exhibited 1.5 times differences in motion in cage and motion duration as well (FIG. 12 and FIG. 13).

Hyponeophagia

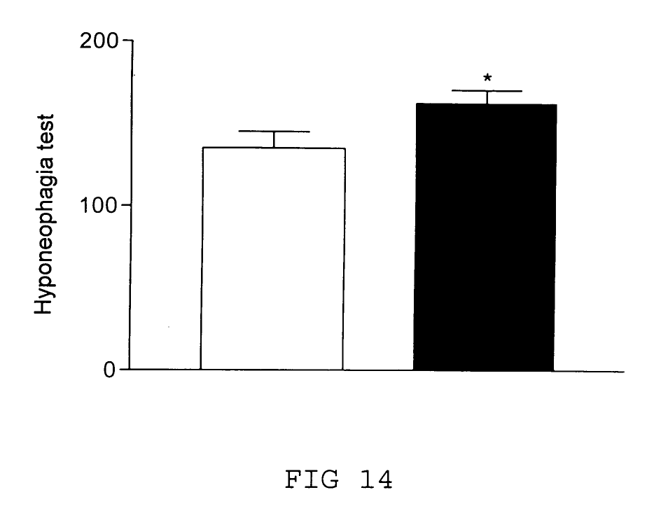

Mice starved for 24 hours were examined for the rapidity of acceptance of unknown food in a novel environment and if they accept it at all. No difference was observed between male mice, while female mice with Wfs1 gene deficiency largely avoided novel food. Comparison of wild type and genetically deficient female mice gave a statistically significant difference (FIG. 14).

Localisation in Brain

The expression profile of β-galactosidase refers to preferred expression of the Wfs1 gene in brain structures related to olfactory sensation and emotions. Especially remarkable expression of the Wfs1 gene is observed in two most important brain structures related to anxiety—the central nucleus of amygdala and the bednucleus of stria terminalis. The role of nucelus accumbens is remarkable in explorative behaviour as well and also in this structure very selective and remarkable expression of the Wfs1 gene was observed. The selective expression of the Wfs1 gene in the CA1 region of hippocampus is also worth attention.

These results obtained from morphological studies are an important support to the changes described in behavioural experiments.

Mice with Wfs1 gene deficiency (especially females) exhibit a very important adaptational disturbance in new environment, which is apparently caused by changes in the limbic structures of the brain (amygdala, bednucleus of stria terminalis and accumbens).

### Example 3

### Identification of Compounds Suitable for the Treatment of Diseases or Conditions Caused by Pathological Anxiety

The following example describes one possible mode for using the invention for the identification of compounds suitable for the treatment of diseases or conditions caused by pathological anxiety.

Two groups of mice with Wfs1 gene deficiency took part in the experiment. Mice in the test group were injected a solution containing a compound or a mixture of compounds in a known concentration. The administration was performed into the abdominal cavity, when the agent was capable to penetrate effectively the haematoencephal barrier (e.g. a low-molecular compound) or into brain ventricles, when the agent (e.g. a peptide or other rapidly metabolized compound) was not able to penetrate it. The mice in the control group were administred physiological saline. After the administration of the agent or physiological saline a behavioural experiment was performed on the animals, where their anxiety behaviour was assessed in elevated plus-maze. The agent was considered as reducing anxiety, if the anxiety behaviour of the animals in test group was statistically significantly reduced in comparison with the mice of the control group (FIG. 4, FIG. 5 and FIG. 6).

Diazepam in the dose of 1 mg/kg was used for testing agents, but the current invention is not limited to the named compound. The high efficiency of diazepam suggests that the named transgenic mouse can be utilized for screening for new potential anxiolytic drugs.

### Example 4

### Using the Wfs1 Protein as a Target for Identification of the Compounds with an Effect Against Pathological Anxiety

The following example describes one possible mode for using the wfs1 protein of the invention as a target for the identification of compounds with an effect against pathological anxiety or anxiety disorders.

Two groups of mice with Wfs1 gene deficiency took part in the experiment. Mice in the test group were injected a solution containing a compound or a mixture of compounds in a known concentration, whereas the compound exhibits direct or indirect effect on the expression and biological activity of the Wfs1 protein. The administration was performed into the abdominal cavity, when the agent was capable to penetrate effectively the haematoencephal barrier (e.g. a low-molecular compound) or into brain ventricles, when the agent (e.g. a peptide or other rapidly metabolized compound) was not able to penetrate the biological barrier between brain and blood. The mice in the control group were administred physiological saline or the solvent of the solution of the tested compound. After the administration of the agent or solvent a behavioural experiment was performed on the animals, where their anxiety behaviour was assessed in elevated plus-maze. The agent was considered as reducing anxiety, if the anxiety behaviour of the animals in test group was statistically significantly reduced in comparison with the mice of the control group.

Diazepam in the dose of 1 mg/kg was used for testing agents, but the current invention is not limited to the named compound.

Assessment of the agents increasing the expression of the Wfs1 protein.

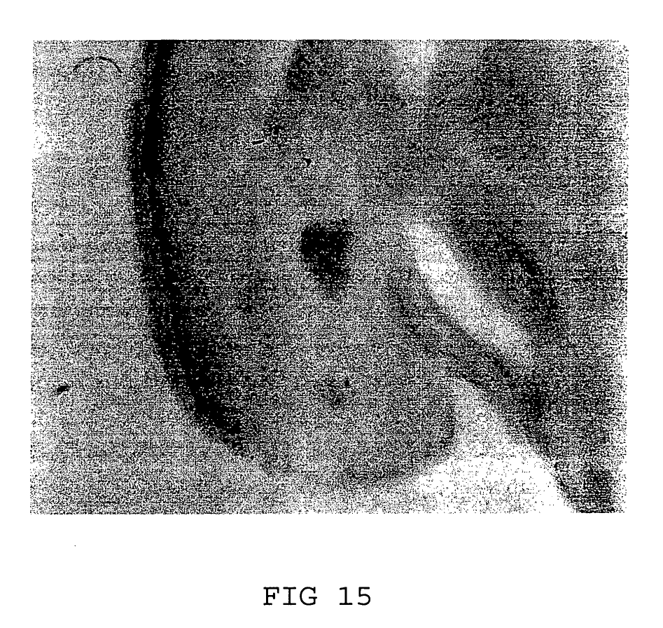

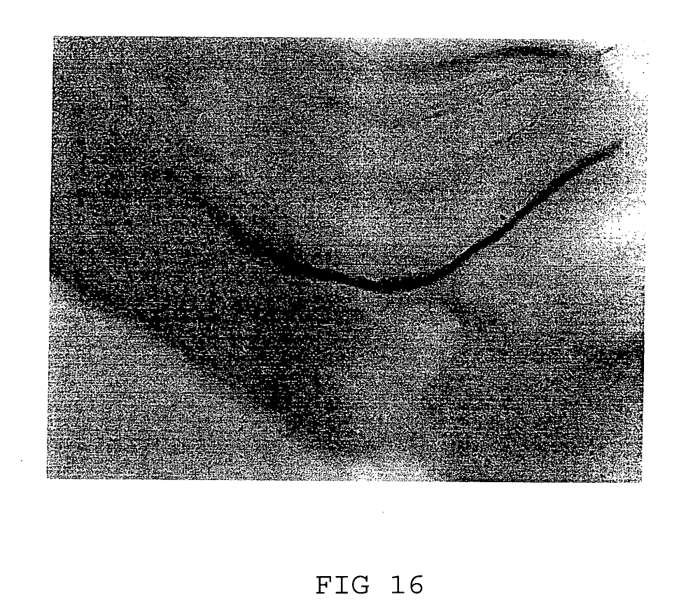

As one aspect of the invention, the described animal model was used for assessing the compounds (agents), which increase the expression of the Wfs1 protein. The current animal model expressed a compound LacZ-Wfs1 protein lacking the activity of the Wfs1 protein enabling the assessment of the level of expression of the Wfs1 protein in an animal who lacked the functional Wfs1 protein by measuring the activity of the lacZ protein directly in whole organs and tissues of the animal after fixation with paraformaldehyde. The exemplary staining procedure used in the current invention for the identification of the Wfs1-NLSLacZ compound protein in Wfs1 deficient mice comprised: 1) Wfs1 −/− mice were deeply insensitized with ketamine and fixed by transcardiac perfusion with 20 mL PBS and 20 mL 2% PFA; 2) Organs of interest were dissected, embedded in 30% sucrose solution, slices and incubated for 24 hours in lacZ staining solution (5 mM K3Fe(CN)6; 5 mM K4Fe(CN)6; 1 mg/mL X-Gal; 0.125% IGEPAL in 0.1M PB, pH 7.3) at ambient temperature in dark; 3) samples were recorded with Canon digital CCD camera, the images were processed with Adobe Photoshop software. The results are shown in FIG. 15 and FIG. 16.

It appears from these figures, that using the present invention it is possible to check and measure the expression of the Wfs1 protein, i.e. the expression after translation, applying shown simple method of staining. This method is significantly more informative from the functional aspect than just sole measurement of transcriptional activity (assessment of mRNA level), as it enables the assessment of changes in proteins. The activation of the function of the Wfs1 protein gives rise to enhanced lacZ staining. This example also describes how to assess changes in the expression of the Wfs1 protein in various brain regions, when the effects of a tested medication are described. This is a feature of especially high practical value, as anti-anxiety substances presumably possess specific effect in various brain structures. Localization of effects in this manner helps to develop drugs with less undesired side effects.

1. An animal model for pathological anxiety, which comprises a rodent with disabled function of a Wfs1 gene, wherein said Wfs1 gene encodes for Wfs1 protein.

2. An animal model according to claim 1, wherein said rodent is mouse lacking both wild type alleles of the Wfs1 gene.

3. An animal model according to claim 1, wherein a DNA fragment encoding an identifiable marker protein is inserted into the coding sequence of the Wfs1 gene of the rodent.

4. An animal model according to claim 1, wherein expression of the Wfs1 protein of the rodent has been reduced compared to a wild type rodent.

5. A method for identifying compounds suitable for the treatment of diseases or conditions caused by pathological anxiety, wherein the named method comprises:
a) administering one or more agents under investigation to the animal model of claim 1; and
b) assessing whether at least one marker of anxiety are reduced by administration of said one or more agents under investigation.

6. A method for identifying compounds with effects against pathological anxiety using Wfs1 protein as target, comprising:
a) administering one or more agents under investigation to a rodent lacking the Wfs1 protein or to a rodent with reduced levels of a functional Wfs1 protein; and
b) assessing whether a level of expression of the Wfs1 protein has increased by administration of said one or more agents under investigation; and
c) determining if symptoms of anxiety have decreased or been eliminated by administration of said one or more agents under investigation.
